(19) United States
(12) Patent Application Publication
(10) Pub. No.: US 2010/0229256 A1
(43) Pub. Date: Sep. 9, 2010

(54) PROPAGATION OF TRANSGENIC PLANTS

(75) Inventors: Mariya Somleva, Cambridge, MA (US); Aminat Ali, Cambridge, MA (US)

Correspondence Address: Pabst Patent Group LLP, 1545 PEACHTREE STREET NE, SUITE 320, ATLANTA, GA, 30309, US

(73) Assignee: Metabolix, Inc.

(21) Appl. No.: 12/718,559

(22) Filed: Mar. 5, 2010

Related U.S. Application Data
(60) Provisional application No. 61/157,691, filed on Mar. 5, 2009.

(51) Int. Cl. A01H 4/00; A01H 5/00

(52) U.S. Cl. 800/268; 800/298

(57) ABSTRACT

Methods for increasing the product yield from plants, preferably transgenic plants, are provided. It has been discovered that in vitro cultures from donor plants produce plants that have two or three fold increase in product yield. One embodiment provides a method for increasing product yield from a transgenic plant by initiating an in vitro culture from a donor transgenic plant, wherein the donor transgenic plant is genetically engineered to produce a product and regenerating a second transgenic plant from the in vitro culture, wherein the yield of the product from the second transgenic plant is greater than the yield of the product from the donor transgenic plant. In a preferred embodiment, the transgenic plant is a graminaceous plant such as switchgrass.

### CROSS REFERENCE TO RELATED APPLICATION

This application claims the benefit of U.S. Provisional Application No. 61/157,691, filed on Mar. 5, 2009. The entire disclosure of the above application is incorporated herein by reference.

### FIELD OF THE INVENTION

The invention is generally related to plant tissue cultures, transformed plants, and methods for propagating them as well as methods for increasing yield of recombinant products in plants.

### BACKGROUND OF THE INVENTION

Plant regeneration from cells or tissues cultured in vitro is a fundamental requirement for most applications of plant biotechnology, including synthesis of recombinant proteins and industrial raw materials in transgenic plants. Because product yield and concentration are major factors in process economics, improving product accumulation is crucial for enhancing the commercial success of plant-based production systems (Doran, 2006).

Considerable research effort has been made to apply molecular technologies to increase transgene expression, to improve translation and to target products to subcellular locations optimal for their accumulation in plants. Once a suitable transgenic line has been identified then traditional plant breeding techniques including crossing into elite germplasm are used to further improve the levels of the product of interest and develop commercial lines (for review Streatfield, 2007). Traditional plant breeding is an extremely time consuming process requiring several breeding cycles ultimately taking several years. This has been the practice for the current commercial GMO crops such as soybean, cotton, canola and corn where breeding capabilities are highly advanced. For biomass crops such as switchgrass, sugarcane and miscanthus the options for traditional plant breeding methods are more limited and more challenging based on the complicated genetics of these plant species.

As renewable sources of energy and raw materials, agricultural feedstocks based on crops are the ultimate replacement of declining oil reserves. Plants use solar energy and carbon dioxide to make thousands of complex and functional biochemicals beyond the capability of the modern synthetic chemistry. These include fine and bulk chemicals, pharmaceuticals, polymers, resins, food additives, bio-colorants, adhesives, solvents, and lubricants. Genetic engineering of crop plants as production platforms for modified and new materials provides a sustainable solution for high value eco-friendly industrial applications. In addition there is general interest in improving the value of crops by improving the characteristics of crops including: agronomic traits such as yield, nitrogen and water use efficiency; plant composition for example higher or lower lignin content, increased starch content, increased oil content; changing the composition of plant storage materials such as triglycerides or starches; introducing novel co-products such as polyhydroxyalkanoate polymers; and introducing multiple hydrolytic enzymes such as cellulases, xylanases and the like using transgene technologies. This approach requires advances in science and technology to speed up the development of commercial lines expressing single or multiple transgenes.

### SUMMARY OF THE INVENTION

Provided herein are efficient procedures for improving the characteristics of a plant, for example a transgenic plant, by developing an in vitro tissue culture of the plant, maintaining the in vitro tissue culture for a period of time, regenerating plants from the in vitro tissue culture and screening the regenerated plants for a characteristic of interest as compared to the original donor plant. The characteristic of the plant can be any characteristic of interest including but not limited to an increase in biomass, an increase in product production, improved root growth, improved drought resistance, increased fiber strength, increased fiber diameter, improved pest resistance, or increased flower or seed production.

One embodiment provides a method for improving the characteristics of a plant by initiating an in vitro culture from a donor plant, and regenerating a second plant from the in vitro culture, wherein the characteristics of the second plant are different than the characteristics in the donor plant. In a preferred embodiment, the plant is transgenic. Exemplary plants include, but are not limited to graminaceous plants.

When the donor plant is transgenic, the transgenic plant can be a primary transformant. The donor transgenic plant can also be propagated from a primary transformant.

In certain embodiments, the in vitro culture is initiated from transgenic plants grown from seeds obtained from controlled crosses between transgenic plants or between transgenic and non-transgenic, wild type plants or from self-pollinated transgenic plants.

A preferred characteristic to be improved includes, but is not limited to the yield of the product from a transgene. Typically, the yield of a transgene product from the second transgenic plant is at least two fold greater than the donor transgenic plant. In one embodiment, the yield of the product from the second transgenic plant is at least three fold greater than the donor transgenic plant.

Representative plants include switchgrass, miscanthus, sugarcane, corn, arianthus, sorghum, cereals and other forage and turf grasses.

One embodiment provides a method for propagating transgenic plants including culturing immature inflorescence-derived callus cells initiated from transformed graminaceous plants, dividing the callus cells into first and second portions, regenerating transgenic plants from the first portion of the callus cells, propagating the second portion of the callus cells to produce additional callus cells, and continuing until a desired number of regenerated transgenic plants is achieved.

Still another embodiment provides a method for supertransforming transgenic plants by culturing immature inflorescence-derived callus cells initiated from transformed graminaceous plants, transforming the callus cells with vector comprising a transgene different from the transgenes in the donor plant and regenerating a plant from the re-transformed callus cells. The transgene can encode one or more proteins involved in metabolic pathways for the synthesis of products, involved in metabolic pathways for the improvement of plant architecture and biomass yield, involved in metabolic pathways for the modification of the plant cell wall or lignin content and composition, encoding herbicide or pesticide resistance, encoding one or more enzyme activities involved in tolerance to biotic and abiotic stress factors, involved in reduced agronomic inputs, water use efficiency or drought tollerance or in gene containment.

In one embodiment, the in vitro tissue culture is a callus culture.

In a preferred embodiment the callus culture is derived from an in vitro panicle culture which is in turn derived from an immature inflorescence tissue.

In another embodiment the in vitro tissue culture is derived from other meristematic tissues including for example nodal segments.

In another embodiment during the period of maintaining the in vitro tissue culture, the in vitro tissue culture can be subjected to various environmental stress conditions such as varying light intensity, high salt, low nitrogen, low phosphate and the like.

In another embodiment the regenerated plants are screened for an increased level of transgene expression.

In another embodiment the regenerated plants are screened for an increased level of a recombinant product.

In another embodiment the regenerated plants are screened for altered composition or agronomic performance.

Also provided are in vitro propagated transgenic plants that produce higher amounts of genetically engineered products than the donor transgenic plants used to initiate the in vitro cultures.

Methods for rapidly and efficiently expanding the number of plants obtained through in vitro cultures are also provided.

Methods for increasing the product yield from transgenic plants are provided. It has been discovered that the propagation of transgenic plant lines (a donor transgenic plant) in in vitro tissue cultures and then regenerating plants from said cultures enables the identification of transgenic plant lines that have a two or three fold increase in product yield. This completely unexpected result provides a general method for speeding up and improving the characteristics of any transgenic plant for any measurable characteristic as compared to the original transgenic line. In addition this result provides a general tissue culture based method in which the transgenic material can be subjected to different environmental conditions during the tissue culture process, including during the regeneration step, thereby enabling a selection process for regenerated plants with improved characteristics. One embodiment provides a method for increasing product yield from a transgenic plant by initiating an in vitro culture from a donor transgenic plant, wherein the donor transgenic plant is genetically engineered to produce a product and regenerating a second transgenic plant from the in vitro culture, wherein the yield of the product from the second transgenic plant is greater than the yield of the product from the donor transgenic plant. In a preferred embodiment, the transgenic plant is a graminaceous plant such as switchgrass miscanthus, sugarcane, corn, arianthus, sorghum, cereals and other forage and turf grasses. The donor transgenic plant can be a primary transformant or the donor transgenic plant can be propagated from a primary transformant. In certain embodiments, the in vitro culture is initiated from transgenic plants regenerated from immature inflorescence-derived callus cultures or nodal segments from stably transformed plants.

Although polyhydroxyalkanoates are exemplified as the product produced by the transgenic plants, one of skill in the art will recognize that the transgenic plants can be engineered to express any transgene. The polyhydroxyalkanoates can be homo or co-polymers. Exemplary polyhydroxyalkanoates include polyhydroxybutyrate and co-polymers thereof.

Another embodiment provides a method for propagating transgenic plants by culturing immature inflorescence-derived callus cells initiated from transformed graminaceous plants and regenerating transgenic plants from a portion of the callus cells. The callus cells can be subcultured repeatedly to increase the number of regenerated plants derived from the callus cells. In one embodiment, tens, hundreds or even thousands of transgenic plants/g fresh weight callus are produced.

Also provided is a method for re-transforming (supertransforming) transgenic plants by culturing immature inflorescence-derived callus cells initiated from graminaceous plants transformed with a first vector, and re-transforming the callus cells with a second vector having the same and/or additional transgenes and producing callus cells with two different sets of transgenes. The method also includes regenerating a plant from the re-transformed callus cells. Preferably the transformed graminaceous plant is selected from switchgrass, miscanthus, sugarcane, corn, arianthus, sorghum, cereals and other forage and turf grasses. The second transgene(s) are typically involved in synthesis of recombinant protein(s) or industrial products(s).

Transgenic plants, plant material, plant tissue, and plant parts such as seeds from the transgenic plant produced by the disclosed methods are also provided.

The disclosed plants can be used as a source of biorefinery feedstock.

### BRIEF DESCRIPTION OF THE DRAWINGS

FIG. 1 shows a schematic diagram of the general procedure for in vitro callus culture initiation and plant regeneration from immature inflorescence-derived cultures. 1 Culture initiation and panicle development (3 wk); 2 Callus initiation and growth (3 wk); 3 Plant regeneration (3 wk); 4 Callus propagation (optional) for one to six in vitro cycles (4 wk each); 5 Plant growth (4 wk); 6 PHB content analysis and transfer to soil; 7 Plant regeneration from callus propagated for 2-6 months (optional); 8 Culture initiation (when needed).

FIG. 2 is a graph of number of plants (percent of total) versus in vitro cycles showing a comparison of polyhydroxybutyrate (PHB) production in leaves of transgenic plants in tissue culture regenerated from immature inflorescence-derived callus initiated from line 56-2a-113 and propagated for 1-6 in vitro cycles. The number of plantlets analyzed by GC/MS (n) is indicated above the bars. The two primary transformants from this line contained 0.62% and 0.45% DW (dry weight) PHB in tissue culture.

FIGS. 3A-C are graphs showing polyhydroxybutyrate (PUB) production in plants obtained from immature inflorescence-derived callus cultures initiated from the line 56-2a-1/3. FIGS. 3A and 38 show polymer levels in plants in tissue culture produced from callus propagated for one (A) and six (B) in vitro cycles. FIG. 3C shows a comparison of PHB content in tissue culture and soil in plants obtained after one (plants 1-10) and six in vitro cycles (plants 11-20). T₀: the primary transformant used for callus initiation.

FIGS. 4A-C are graphs showing PHB content in plants obtained from freshly initiated immature inflorescence-derived callus cultures from different transgenic lines. FIGS. 4A and 4B show polymer accumulation in plants in tissue culture obtained from cultures from two high producers, Alamo genotypes 56 (A) and 215 (B). FIG. 4C shows a comparison of PHB content in tissue culture and soil in some of the plants shown in FIG. 4A. T₀: the primary transformants used for callus initiation.

FIGS. 5A and 5B are graphs showing polyhydroxybutyrate (PHB) production in plants obtained from immature inflorescence-derived callus initiated from a transgenic plant propagated from a primary transformant.

FIGS. 5A and 5B show polymer levels in plants in tissue culture produced from callus propagated for one (A) and six (B) in vitro cycles. T₀: a primary transformant from the line 56-2a-1/3 used as a source of immature inflorescences for callus initiation; M: a transgenic plant regenerated from these cultures after four in vitro cycles.

FIGS. 6A and 6B are graphs showing polymer production in plants obtained from node cultures initiated from a primary transformant and from a transgenic plant propagated from the same primary transformant. FIG. 6A shows PHB levels in plants in tissue culture produced from nodal segments from the transgenic line 56-2a-113 and FIG. 6B shows polymer content in plant formed from node cultures initiated from a micropropagated transgenic plant. T₀: a primary transformant from the line 56-2a-1/3 used as a source of nodal segments for culture initiation; M: a transgenic plant regenerated from immature inflorescence-derived callus from line 56-2a-1/3 after six in vitro cycles.

### DETAILED DESCRIPTION OF THE INVENTION

Provided herein are methods for increasing a desired characteristic of a plant, for example product yield from a line of plants, by in vitro selection and methods for propagating plants by culturing immature inflorescence-derived callus cells initiated from the plants. Also provided are methods of supertransforming plants, as well as plants and seeds made by these methods.

### I. DEFINITIONS

Unless otherwise indicated, the disclosure encompasses conventional techniques of plant breeding, microbiology, cell biology and recombinant DNA, which are within the skill of the art. See, e.g., Sambrook and Russell, Molecular Cloning: A Laboratory Manual, 3rd edition (2001); Current Protocols In Molecular Biology [(F. M. Ausubel, et al. eds., (1987)]; Plant Breeding: Principles and Prospects (Plant Breeding, Vol 1) M. D. Hayward, N. O. Bosemark, I. Romagosa; Chapman & Hall, (1993); Coligan, Dunn, Ploegh, Speicher and Wingfeld, eds. (1995) Current Protocols in Protein Science (John Wiley & Sons, Inc.); the series Methods in Enzymology (Academic Press, Inc.): PCR 2: A Practical Approach (M. J. MacPherson, B. D. Hames and G. R. Taylor eds. (1995)].

Unless otherwise noted, technical terms are used according to conventional usage. Definitions of common terms in molecular biology may be found in Lewin, Genes VII, published by Oxford University Press, 2000; Kendrew et al. (eds.), The Encyclopedia of Molecular Biology, published by Wiley-Interscience., 1999; and Robert A. Meyers (ed.), Molecular Biology and Biotechnology, a Comprehensive Desk Reference, published by VCH Publishers, Inc., 1995; Sambrook and Russell. (2001) Molecular Cloning: A Laboratory Manual 3rd. edition, Cold Spring Harbor Laboratory Press.

A number of terms used herein are defined and clarified in the following section.

The term “PHA copolymer” refers to a polymer composed of at least two different hydroxyalkanoic acid monomers. The term “PHA homopolymer” refers to a polymer that is composed of a single hydroxyalkanoic acid monomer.

The term “endogenous” with regard to a nucleic acid refers to nucleic acids normally present in the host.

As used herein, a “vector” is a replicon, such as a plasmid, phage, or cosmid, into which another DNA segment may be inserted so as to bring about the replication of the inserted segment. The vectors can be expression vectors.

As used herein, an “expression vector” is a vector that includes one or more expression control sequences

As used herein, an “expression control sequence” is a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

As used herein, “operably linked” means incorporated into a genetic construct so that expression control sequences effectively control expression of a coding sequence of interest.

As used herein, “transformed” and “transfected” encompass the introduction of a nucleic acid into a cell by a number of techniques known in the art.

“Plasmids” are designated by a lower case “p” preceded and/or followed by capital letters and/or numbers.

As used herein the term “heterologous” means from another host. The other host can be the same or different species.

The term “cell” refers to a membrane-bound biological unit capable of replication or division.

The term “construct” refers to a recombinant genetic molecule including one or more isolated polynucleotide sequences.

Genetic constructs used for transgene expression in a host organism comprise in the 5′-3′ direction, a promoter sequence; a sequence encoding an inhibitory nucleic acid disclosed herein; and a termination sequence. The open reading frame may be orientated in either a sense or anti-sense direction. The construct may also comprise selectable marker gene(s) and other regulatory elements for expression.

The term “plant” is used in it broadest sense. It includes, but is not limited to, any species of woody, ornamental or decorative, crop or cereal, fruit or vegetable plant, and photosynthetic green algae (e.g., Chlamydomonas reinhardtii). It also refers to a plurality of plant cells that are largely differentiated into a structure that is present at any stage of a plant's development. Such structures include, but are not limited to, a fruit, shoot, stem, leaf, flower petal, etc. The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

A non-naturally occurring plant refers to a plant that does not occur in nature without human intervention. Non-naturally occurring plants include transgenic plants and plants produced by non-transgenic means such as plant breeding.

The term “product” refers to a desired biochemical made in the plant, plant tissues and plant cells. The term can include, but is not limited to, fine and/or bulk chemicals, pharmaceuticals, polymers, resins, food additives, biocolorants, adhesives, solvents, lubricants, antibiotics, organic acids, amino acids, proteins, vitamins, polysaccharides, and other compounds capable of being made in plants.

The term “biorefinery feedstock” refers to biomass obtained from plants.

### II. METHODS FOR PROPAGATING GRAMINACEOUS PLANTS

### A. Propagation of Plants

Tissue culture methods for large-scale vegetative propagation of graminaceous plants, in particular, transgenic plants with increased yield of products encoded by the transgenes are provided. A preferred plant is switchgrass. In one embodiment, tissue culture methods are provided to increase production of polyhydroxyalkanoate in switchgrass plants engineered to produce polyhydroxyalkanoate. In a preferred embodiment, switchgrass is transformed with one or more genes encoding enzymes for the production of polyhydroxybutyrate (PHB). One of the advantages of this approach is the ability to use stable transgenic lines with high expression levels of the recombinant genes as a source of explants for culture initiation and plant regeneration.

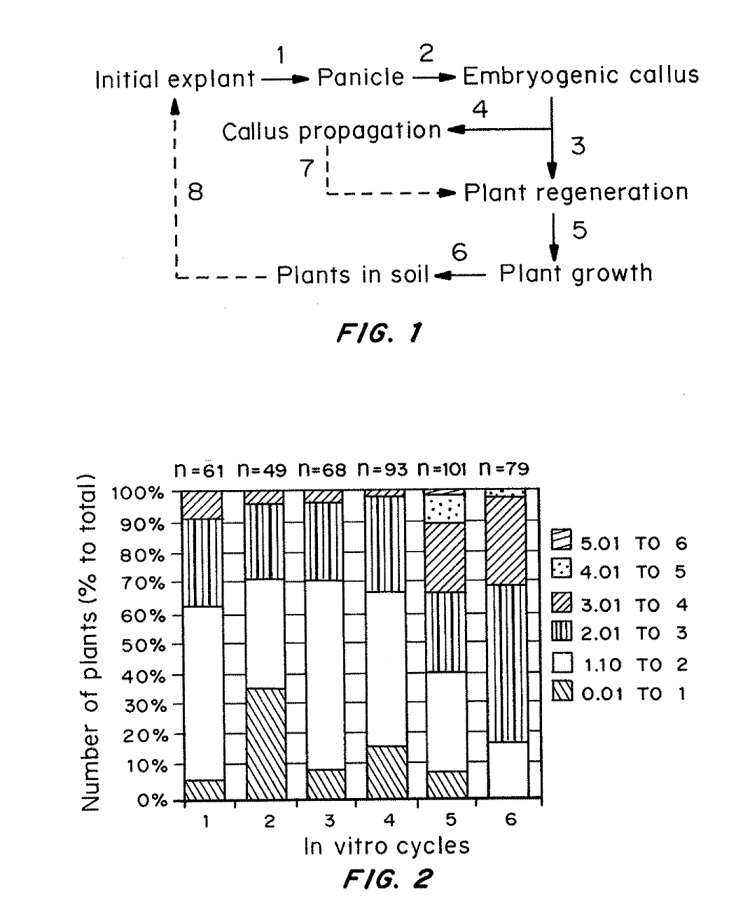

FIG. 1 provides a schematic diagram of the general procedure for callus initiation and plant regeneration from immature inflorescence-derived cultures. A plant, for example, transgenic switchgrass plants, cv. Alamo, carrying the PHB pathway genes driven by the maize cab-m5 promoter (Sullivan et al., 1989; Becker et al., 1992) can be used as starting material. For instance, tillers at the elongation stage of plant development with 2-4 nodes prior to flowering can be used as a starting material. Plant donors for the starting material can be primary transformants obtained from transformed mature caryopsis-derived embryogenic callus cultures, plants regenerated from immature inflorescence-derived embryogenic callus initiated from transgenic plants, plants obtained from nodal segments from transgenic plants, or plants grown from seeds obtained from controlled crosses between transgenic plants or between transgenic and non-transgenic, wild-type, plants.

1. Panicle Formation and Culture Initiation

In vitro developed panicles are obtained from the top culm node of elongating tillers from plants, preferably transformed plants, following a previously published procedure for non-transformed switchgrass plants (Alexandrova et al., 1996a). For callus initiation (FIG. 1, step 2), individual spikelets from panicles formed in tissue culture are plated on MS medium for callus initiation and growth (Denchev and Conger, 1994). Resultant embryogenic callus cultures are cultured at 28° C., in the dark and propagated by monthly transfers on to a fresh medium (Somleva, 2006).

Plants are also regenerated from in vitro cultured culm nodes of elongating tillers from transgenic plants following an established protocol for culture initiation from non-transformed switchgrass plants (Alexandrova et al., 1996b) and cultured at 28° C. with a 16 h photoperiod (cool white fluorescent bulbs, 80 μmol/m²/s).

2. Callus Propagation and Plant Regeneration:

Immature inflorescence-derived callus cultures are propagated by transferring onto a fresh medium for callus growth (Denchev and Conger, 1994) every four weeks (in vitro cycles) (FIG. 1, step 4). Most of the callus cultures initiated from different donor plants are maintained for 6 months in total (six in vitro cycles) (FIG. 1, step 7).

For plant regeneration (FIG. 1, step 3), pieces of callus are pre-weighed and plated on MS medium for plant regeneration (Denchev and Conger, 1994). Cultures are incubated at 28° C. with a 16 h photoperiod (cool white fluorescent bulbs, 80 μmol/m²/s) for four weeks followed by a transfer onto a fresh regeneration medium for another four weeks (FIG. 1, step 5). The plants can then be transferred to soil (FIG. 1, step 6).

As described in the Examples, all of the in vitro propagated plants were transgenic and most of them accumulated polymer at levels similar or higher then the donor lines. In tissue culture, PHB production was analyzed in more than 1,200 transgenic plants generated from immature inflorescence-derived callus. About 58% of them contained polymer at levels higher than the corresponding primary transformant used for culture initiation. This number varied in different lines. About 70-96% of the regenerants obtained from callus cultures initiated from 3 of the best producers among the primary transformants contained PHB at higher levels (up to 5.25% DW) than the donor plants at the same stage. In soil, some of these mieropropagated plants produced the highest polymer levels (up to 6.09% DW) ever measured in switchgrass or other graminaceous species, such as corn (5.7% DW PUB, Poirier and Gruys, 2002) and sugarcane (1.8-1.9% DW PHB, Purnell et al., 2007; Petrasovich et al., 2007).

Polymer accumulation was found to vary between the regenerants propagated from different lines. In certain embodiments, the cultured transgenic plants produced two-fold and even three fold more product than the donor plant. However, variability also existed within plants originated from the same line. For example, plants cloned from the line 56-2a-1/3 through node cultures possessed PHB levels in the wide range from 0.34% DW to 4.92% DW. Generally, a wide range of product accumulation is often seen with genetically engineered quantitative traits. In plants propagated through immature inflorescence-derived cultures, no clear correlation was found between the PHB production and the duration of callus propagation. High producers were identified in populations of plants propagated from freshly initiated cultures as well as from callus cultures propagated for longer periods of time, up to six months. More importantly, even plants accumulating more than 5-6% DW PHB grew and developed normally in a greenhouse, although the donor Tₒ plant (2.56% DW PHB in soil) showed slight chlorosis and growth reduction (Somleva et al., 2008). These findings suggest that if any somaclonal changes have occurred in the cultured tissues, they appear to favor the increased PHB production and improved agronomic performance of the in vitro propagated plants.

### B. Rapid Generation of High Numbers of Plants

Another advantage of the disclosed procedure is the generation of large numbers of transgenic plants as described in the Examples. More than 30,500 PHB producing plants were obtained from 14 transgenic lines. About 20% of these plants were obtained from freshly initiated callus within 3 months after culture initiation. The rest of the propagated plants were produced from immature inflorescence callus maintained for 2-6 in vitro cycles. Another 11,767 plants were obtained from cultures initiated from transgenic plants derived from primary transformants. In addition, 218 plants were cloned from node cultures from different types of donor plants. All of the transgenic switchgrass plants propagated through immature inflorescence-derived callus or node cultures appeared morphologically normal and uniform in their growth under in vitro and greenhouse conditions.

### III. RE-TRANSFORMANTS (SUPERTRANSFORMANTS)

The immature inflorescence-derived callus cultures from transgenic plants can also be used as a target material for introduction of additional recombinant genes for combining or reinforcing of engineered traits into transgenic lines with desired characteristics. This approach could be used for engineering of new metabolic pathways (e.g., synthesis of PHS and other value-added co-products) and for manipulations of the metabolite flux through competing and interconnected pathways. Genes for improved plant architecture and biomass yield, modified lignin content and composition, herbicide resistance, biotic and abiotic stress tolerance, and gene containment can also be introduced into these cultures.

### IV. METHODS FOR PRODUCING TRANSFORMED PLANTS

### A. Vectors and Constructs

Vectors and constructs for expressing a transgene in plants, particularly graminaceous plants are well known in the art. The constructs can include an expression cassette containing one or more transgenes, for example enzymes that can provide desired input or output traits to a plant. Transformation constructs can be engineered such that transformation of the nuclear genome and expression of transgenes from the nuclear genome occurs. Alternatively, transformation constructs can be engineered such that transformation of the plastid genome and expression of the plastid genome occurs.

An exemplary construct contains operatively linked in the 5′ to 3′ direction, a promoter that directs transcription of a nucleic acid sequence, a nucleic acid sequence encoding a protein of interest, and a 3′ polyadenylation signal sequence.

Generally, nucleic acid sequences encoding proteins or interest are first assembled in expression cassettes behind a suitable promoter expressible in plants. The expression cassettes may also include any further sequences required or selected for the expression of the transgene. Such sequences include, but are not restricted to, transcription terminators, extraneous sequences to enhance expression such as introns, vital sequences, and sequences intended for the targeting of the gene product to specific organelles and cell compartments. These expression cassettes can then be easily transferred to the plant transformation vectors. Representative plant transformation vectors are described in plant transformation vector options available (Gene Transfer to Plants (1995), Potrykus, I. and Spangenberg, G. eds. Springer-Verlag Berlin Heidelberg New York; “Transgenic Plants: A Production System for Industrial and Pharmaceutical Proteins” (1996), Owen, M. R. L. and Pen, J. eds. John Wiley & Sons Ltd. England and Methods in Plant Molecular biology-a laboratory course manual (1995), Maliga, P., Klessig, D. F., Cashmore, A. R., Gruissem, W. and Varner, J. E. eds. Cold Spring Laboratory Press, New York). An additional approach is to use a vector to specifically transform the plant plastid chromosome by homologous recombination (U.S. Pat. No. 5,545,818 to McBride et al.), in which case it is possible to take advantage of the prokaryotic nature of the plastid genome and insert a number of transgenes as an operon.

In a preferred embodiment, the transgenes encode enzymes and other factors required for production of a biopolymer, such as a polyhydroxyalkanoate (PHA).

The following is a description of various components of typical expression cassettes.

1. Promoters

The selection of the promoter used in expression cassettes determines the spatial and temporal expression pattern of the transgene in the transgenic plant. Selected promoters express transgenes in specific cell types (such as leaf epidermal cells, mesophyll cells, root cortex cells) or in specific tissues or organs (roots, leaves, seeds, or flowers, for example) and the selection reflects the desired location of accumulation of the gene product. Alternatively, the selected promoter drives expression of the gene under various inducing conditions.

Promoters vary in their strength, i.e., ability to promote transcription. Depending upon the host cell system utilized, any one of a number of suitable promoters known in the art may be used. For example, for constitutive expression, the CaMV 35S promoter, the rice actin promoter, or the ubiquitin promoter may be used. For example, for regulatable expression, the chemically inducible PR-1 promoter from tobacco or Arabidopsis may be used (see, e.g., U.S. Pat. No. 5,689,044 to Ryals et al.).

Suitable category of promoters are wound inducible promoters. Numerous promoters have been described which are expressed at wound sites. Preferred promoters of this kind include those described by Stanford et al. Mol, Gen. Genet. 215: 200-208 (1989), Xu et al. Plant Molec. Biol. 22: 573-588 (1993), Logemann et al. Plant Cell 1: 151-158 (1989), Rohrmeier & Lehle, Plant Molec. Biol. 22: 783-792 (1993), Firek et al. Plant Molec. Biol. 22: 129-142 (1993), and Warner et al. Plant J. 3: 191-201 (1993).

Suitable tissue specific expression patterns include green tissue specific, root specific, stem specific, seed specific, and flower specific. Promoters suitable for expression in green tissue include many which regulate genes involved in photosynthesis, and many of these have been cloned from both monocotyledons and dicotyledons. A suitable promoter is the maize PEPC promoter from the phosphoenol carboxylase gene (Hudspeth & Grula, Plant Molec. Biol. 12: 579-589 (1989)). A suitable promoter for root specific expression is that described by de Framond (FEBS 290: 103-106 (1991); EP 0 452 269 to de Framond) and a root-specific promoter is that from the T-1 gene. A suitable stem specific promoter is that described in U.S. Pat. No. 5,625,136 and which drives expression of the maize trpA gene.

2. Transcriptional Terminators

A variety of transcriptional terminators are available for use in expression cassettes. These are responsible for the termination of transcription beyond the transgene and its correct polyadenylation. Appropriate transcriptional terminators are those that are known to function in plants and include the CaMV 35S terminator, the tm1 terminator, the nopaline synthase terminator and the pea rbcS E9 terminator. These are used in both monocotyledonous and dicotyledonous plants.

A polyadenylation signal can be engineered at the extreme 3′ end of transcript. A polyadenylation signal refers to any sequence that can result in polyadenylation of the mRNA in the nucleus prior to export of the mRNA to the cytosol, such as the 3′ region of nopaline synthase (Bevan, M. et al., Nucleic Acids Res., 11, 369-385 (1983)).

3. Sequences for the Enhancement or Regulation of Expression

Numerous sequences have been found to enhance gene expression from within the transcriptional unit and these sequences can be used in conjunction with the genes to increase their expression in transgenic plants. For example, various intron sequences such as introns of the maize Adh1 gene have been shown to enhance expression, particularly in monocotyledonous cells. In addition, a number of non-translated leader sequences derived from viruses are also known to enhance expression, and these are particularly effective in dicotyledonous cells.

4. Coding Sequence Optimization

The coding sequence of the selected gene may be genetically engineered by altering the coding sequence for optimal expression in the crop species of interest. Methods for modifying coding sequences to achieve optimal expression in a particular crop species are well known (see, e.g. Perlak et al., Proc. Natl. Acad. Sci. USA 88: 3324 (1991); and Koziel et al., Biotechnol. 11: 194 (1993)).

5. Targeting Sequences

The disclosed vectors and constructs may further include, within the region that encodes the protein to be expressed, one or more nucleotide sequences encoding a targeting sequence. A “targeting” sequence is a nucleotide sequence that encodes an amino acid sequence or motif that directs the encoded protein to a particular cellular compartment, resulting in localization or compartmentalization of the protein. Presence of a targeting amino acid sequence in a protein typically results in translocation of all or part of the targeted protein across an organelle membrane and into the organelle interior. Alternatively, the targeting peptide may direct the targeted protein to remain embedded in the organelle membrane. The “targeting” sequence or region of a targeted protein may contain a string of contiguous amino acids or a group of noncontiguous amino acids. The targeting sequence can be selected to direct the targeted protein to a plant organelle such as a nucleus, a microbody (e.g., a peroxisome, or a specialized version thereof, such as a glyoxysome), an endoplasmic reticulum, an endosome, a vacuole, a plasma membrane, a cell wall, a mitochondrion, a chloroplast or another type of plastid. A chloroplast targeting sequence is any peptide sequence that can target a protein to the chloroplasts or plastids, such as the transit peptide of the small subunit of the alfalfa ribulose-biphosphate carboxylase (Khoudi et al., Gene, 197:343-351 (1997)). A peroxisomal targeting sequence refers to any peptide sequence, either N-terminal, internal, or C-terminal, that can target a protein to the peroxisomes, such as the plant C-terminal targeting tripeptide SKL (Banjoko, A. & Trelease, R. N. Plant Physiol., 107:1201-1208 (1995); T. P. Wallace et al., “Plant Organellular Targeting Sequences,” in Plant Molecular Biology, Ed. R. Croy, BIOS Scientific Publishers Limited (1993) pp. 287-288, and peroxisomal targeting in plant is shown in M. Volokita, The Plant J., 361-366 (1991)).

B. Plants and Tissues for Transfection

Both dicotyledons and monocotyledons can be used in the disclosed positive selection system. Preferred host plants are graminaceous plants.

Representative plants useful in the methods disclosed herein include the Brassica family including napus, rappa, sp. carinata and juncea; industrial oilseeds such as Camelina sativa, Crambe, Jatropha, castor, Arabidopsis thaliana, maize, soybean, cottonseed, sunflower, palm, coconut, safflower, peanut, mustards including Sinapis alba, sugarcane and flax. Crops harvested as biomass, such as silage corn, alfalfa, switchgrass, Miscanthus, sorghum or tobacco, also are useful with the methods disclosed herein. Representative cells and tissues for transformation using these vectors include protoplasts, cells, callus tissue, leaf discs, pollen, anthers, and meristems. Algae can also be used. Representative species of algae include, but are not limited to Emiliana huxleyi, Arthrospira platensis (Spirulina), Haematococcus pluvialis, Dunaliella salina, and Chlamydomanas reinhardii.

### C. Transgenes

Although genes encoding enzymes in the PHA pathway are exemplified, it will be appreciated that the disclosed methods for increasing transgene expression can be applied to transgenic plants expressing any transgene. Generation of transgenic plants with modified architecture and development characteristics has been described (WO/2002/033091 A1; WO/2001/090388 A1; WO/2001/036444 A1). Overexpression of exogenous and/or endogenous genes resulting in the improvement of plant growth and biomass yield has been reported (U.S. Pat. No. 7,663,027; WO/2008/049183; WO/2004/106531 A1; WO/2000/0569). Improved vegetative growth and productivity has also been registered in transgenic crops with modified carbon and/or nitrogen metabolism (U.S. Pat. No. 7,012,171; U.S. Pat. No. 7,329,727), delayed leaf senescence (U.S. Pat. No. 7,060,875) or flowering (WO/2002/033091 A1).

Genes involved in defense responses of different organisms have been isolated and used for crop engineering for tolerance to pest infestations and pathogen attacks (U.S. Pat. No. 7,576,261; WO/1995/006730 A1; Soleron et al., Science, 318:1640-1642 (2007)). Plant genes conferring resistance to abiotic stresses have also been identified and introduced in to model and crop plants to improve their tolerance to drought and excessive temperatures (WO/2004/044207 A1; WO/2002/020791 A1; WO/2000/073475 A1).

Genetic modifications of the plant cell wall biosynthesis and composition are very important for the development of lignocellulosic bioenergy platforms (Harris & DeBolt, Plant Biotech J 8:244-262 (2010); Hisano et al. In Vitro Cell. Dev. Biol. 45:306-313 (2009); WO/1998/055596 A1).

Gene technologies allowing for the production of transgenic plants without selectable markers and for containing transgenes among plants have also been developed (Gidoni et al. In Vitro Cell. Dev. Biol. 44:457-467 (2008); Daniell H., Nat Biotechnol, 20:581-586 (2002); U.S. Pat. No. 7,238,853).

The expression of multiple enzymes is useful for altering the metabolism of plants to increase, for example, the levels of nutritional amino acids (Falco et al. Biotechnology 13: 577 (1995)), to modify lignin metabolism (Baucher et al. Crit. Rev. Biochem. Mol. Biol. 38: 305-350 (2003)), to modify oil compositions (Drexler et al. J. Plant Physiol. 160: 779-802 (2003)), to modify starch, or to produce polyhydroxyalkanoate polymers (Huisman and Madison, Microbiol Mol. Biol. Rev. 63: 21-53 (1999). In preferred embodiments, the product of the transgenes is a biopolymer, such as a polyhydroxyalknaoate (PHA), a vegetable oil containing fatty acids with a desirable industrial or nutritional profile, or a nutraceutical compound. The transgene can encode a protein that promotes pest resistance, drought resistance, or plant growth.

In a preferred embodiment, the products of the transgenes are enzymes and other factors required for production of a biopolymer, such as a polyhydroxyalkanoate (PHA).

The transgenes can encode enzymes such as beta-ketothiolase, acetoacetyl-CoA reductase, PHB (“short chain”) synthase, PHA (“long chain”) synthase, threonine dehydratase, dehydratase such as 3-OH acyl ACP, isomerase such as Δ3-cis, Δ2-trans isomerase, propionyl-CoA synthetase, hydroxyacyl-CoA synthetase, hydroxyacyl-CoA transferase, thioesterase, fatty acid synthesis enzymes and fatty acid beta-oxidation enzymes. Useful genes are well known in the art, and are disclosed, for example, by Snell and Peoples Metab. Eng. 4: 29-40 (2002) and Bohmert et. al. in Molecular Biology and Biotechnology of Plant Organelles. H. Daniell, C. D. Chase Eds. (Kluwer Academic Publishers, Netherlands; 2004, pp. 559-585).

PHA Synthases

Examples of PHA synthases include a synthase with medium chain length substrate specificity, such as phaC1 from Pseudomonas oleovorans (WO 91/00917; Huisman, et al. J. Biol. Chem. 266, 2191-2198 (1991)) or Pseudomonas aeruginosa (Timm, A. & Steinbuchel, A. Eur. J. Biochem. 209: 15-30 (1992)), the synthase from Alcaligenes eutrophus with short chain length specificity (Peoples, O. P. & Sinskey, A. J. J. Biol. Chem. 264:15298-15303 (1989)), or a two subunit synthase such as the synthase from Thiocapsa pfennigii encoded by phaE and phaC (U.S. Pat. No. 6,011,144). Other useful PHA synthase genes have been isolated from, for example, Aeromonas caviae (Fukui & Doi, J. Bacteriol. 179: 4821-30 (1997)), Rhodospirillum rubrum (U.S. Pat. No. 5,849,894), Rhodococcus ruber (Pieper & Steinbuechel, FEMS Microbial. Lett. 96(1): 73-80 (1992)), and Nocardia corallina (Hall et. al., Can. J. Microbial. 44: 687-91 (1998)). PHA synthases with broad substrate specificity useful for producing copolymers of 3-hydroxybutyrate and longer chain length (from 6 to 14 carbon atoms) hydroxyacids have also been isolated from Pseudomonas sp. A33 (Appl. Microbial. Biotechnol. 42: 901-909 (1995)) and Pseudomonas sp. 61-3 (Kato, et al. Appl. Microbiol. Biotechnol. 45: 363-370 (1996)).

A range of PHA synthase genes and genes encoding additional metabolic steps useful in PHA biosynthesis are described by Madison and Huisman. Microbiology and Molecular Biology Reviews 63:21-53 (1999)).

Enoyl-CoA Hydratase

The phaJ gene encoding an(R)-specific enoyl-CoA hydratase genes are also well known in the art and include the phaJ gene first isolated form Areomonas caviae (Fukui and Doi, J. Bacteriol. 179: 4821-30 (1997)) and numerous homologs isolated from a wide range of bacteria including Pseudomonas aeruginosa which has four such genes, phaJ1-phaJ4 (Davis, r. et. Al., 2007 Antonie van Leeuwenhoek, 93: 285-296.)

R-3-hydroxyacyl-ACP:CoA Transferase

An R-3-hydroxyacyl-ACP:CoA transferase (PhaG) refers to an enzyme that can convert R-3-hydroxyacyl-ACP, an intermediate in fatty acid biosynthesis, to R-3-hydroxyacyl-CoA, the monomer unit for PHA synthase and thus PHA synthesis. Genes encoding PhaG enzymes have been isolated from a range of Pseudomads, including Pseudomonas putida (Rehm et al., J. Biol. Chem., 273:24044-24051 (1998)), Pseudomonas aeruginosa (Hoffmann et al., FEMS Microbiology Letters, 184, 253-259 (2000)), and Pseudomonas sp. 61-3 (Matsumoto et al, Biomacromolecules, 2:142-147 (2001)). While it has been reported that PhaG can catalyze the complete conversion of R-3-hydroxyacyl-ACP to R-3-hydroxyacyl-CoA in Pseudomonads, in E. coli it has been shown that an additional acyl CoA synthetase activity is needed to accumulate medium chain length PHAs from simple carbon sources in strains engineered to express a medium chain length synthase (US Patent Application 2003/0017576).

Acyl CoA Synthetase

An acyl CoA synthetase refers to an enzyme that can convert free fatty acids, including R-3-hydroxyalkanoic acids, to the corresponding acyl-CoA. Genes encoding acyl CoA synthetases have been isolated from a range of organisms, including the alkK gene from Pseudomonas oleovorans (van Beilen, J. et al. Mol Microbial, 6, 3121-36 (1992)), the fadD gene from E. coli (Black, P. et al., Biol. Chem. 267, 25513-25520 (1992)), and the ydiD gene from E. coli (Campbell et al., Mol Microbial. 47, 793-805 (2003)).

Reductases

A reductase refers to an enzyme that can reduce β-ketoacyl CoAs to R-3-OH-acyl CoAs, such as the NADH dependent reductase from Chromatium vinosum (Liebergesell, M., & Steinbuchel, A. Eur. J. Biochem. 209: 135-150 (1992)), the NADPH dependent reductase from Alcaligenes eutrophus (Peoples, O. P. & Sinskey, A. J. J. Biol. Chem. 264: 15293-15297 (1989))), the NADPH reductase from Zooglaea ramigera (Peoples, O. P. & Sinskey, A. J. Molecular Microbiology 3: 349-357 (1989)) or the NADPH reductase from Bacillus megaterium (U.S. Pat. No. 6,835,820).

Thiolases

A beta-ketothiolase refers to an enzyme that can catalyze the conversion of acetyl CoA and an acyl CoA to a β-ketoacyl CoA, a reaction that is reversible. An example of such thiolases are PhaA from Alcaligenes eutropus (Peoples, O. P. & Sinskey, A. J. J. Biol. Chem. 264: 15293-15297 (1989)), and BktB from Alcaligenes eutrophus (Slater et al. J Bacteriol. 180(8):1979-87 (1998)). An acyl CoA oxidase refers to an enzyme capable of converting saturated acyl CoAs to Δ2 unsaturated acyl CoAs. Examples of acyl CoA oxidases are PDX1 from Saccharomyces cerevisiae (Dmochowska et al. Gene, 1990, 88, 247-252) and ACX1 from Arabidopsis thaliana (Genbank Accession # AF057044).

Acyl CoA Transferase

Acyl CoA-transferase genes and enzymes are described by Huisman et al. (U.S. Pat. No. 7,229,804), Söhling and Gottschalk (J. Bacterial. 178:871-880 (1996)), and Eikmanns and Buckel (Biol. Chem. Hoppe-Seyler 371:1077-1082 (1990)).

### D. Plant Transformation Techniques

1. Nuclear Transformation

The transformation of suitable agronomic plant hosts using vectors expressing transgenes can be accomplished with a variety of methods and plant tissues. Representative transformation procedures include Agrobacterium-mediated transformation, biolistics, microinjection, electroporation, polyethylene glycol-mediated protoplast transformation, liposome-mediated transformation, and silicon fiber-mediated transformation (U.S. Pat. No. 5,464,765 to Coffee, et al.; “Gene Transfer to Plants” (Potrykus, et al., eds.) Springer-Verlag Berlin Heidelberg New York (1995); “Transgenic Plants: A Production System for Industrial and Pharmaceutical Proteins” (Owen, et al., eds.) John Wiley & Sons Ltd. England (1996); and “Methods in Plant Molecular Biology: A Laboratory Course Manual” (Maliga et al. eds.) Cold Spring Laboratory Press, New York (1995)).

Soybean can be transformed by a number of reported procedures (U.S. Pat. Nos. 5,015,580 to Christou, et al.; 5,015,944 to Bubash; 5,024,944 to Collins, et al.; 5,322,783 to Tomes et al.; 5,416,011 to Hinchee et al.; 5,169,770 to Chee et al.).

A number of transformation procedures have been reported for the production of transgenic maize plants including pollen transformation (U.S. Pat. No. 5,629,183 to Saunders et al.), silicon fiber-mediated transformation (U.S. Pat. No. 5,464,765 to Coffee et al.), electroporation of protoplasts (U.S. Pat. Nos. 5,231,019 Paszkowski et al.; 5,472,869 to Krzyzek et al.; 5,384,253 to Krzyzek et al.), gene gun (U.S. Pat. Nos. 5,538,877 to Lundquist et al. and 5,538,880 to Lundquist et al.), and Agrobacterium-mediated transformation (EP 0 604 662 A1 and WO 94/00977 both to Hiei Yukou et al.). The Agrobacterium-mediated procedure is particularly preferred as single integration events of the transgene constructs are more readily obtained using this procedure which greatly facilitates subsequent plant breeding. Cotton can be transformed by particle bombardment (U.S. Pat. Nos. 5,004,863 to Umbeck and 5,159,135 to Umbeck). Sunflower can be transformed using a combination of particle bombardment and Agrobacterium infection (EP 0 486 233 A2 to Bidney, Dennis; U.S. Pat. No. 5,030,572 to Power et al.). Flax can be transformed by either particle bombardment or Agrobacterium-mediated transformation. Switchgrass can be transformed using either biolistic or Agrobacterium mediated methods (Richards et al. Plant Cell Rep. 20: 48-54 (2001); Somleva et al. Crop Science 42: 2080-2087 (2002)). Methods for sugarcane transformation have also been described (Franks & Birch Aust. J. Plant Physiol. 18, 471-480 (1991); WO 2002/037951 to Elliott, Adrian, Ross et al.).

Recombinase technologies which are useful in practicing the current invention include the cre-lox, FLP/FRT and Gin systems. Methods by which these technologies can be used for the purpose described herein are described for example in (U.S. Pat. No. 5,527,695 to Hodges et al.; Dale and Ow, Proc. Natl. Acad. Sci. USA, 88:10558-10562 (1991); Medberry et al., Nucleic Acids Res., 23: 485-490 (1995)).

Engineered minichromosomes can also be used to express one or more genes in plant cells. Cloned telomeric repeats introduced into cells may truncate the distal portion of a chromosome by the formation of a new telomere at the integration site. Using this method, a vector for gene transfer can be prepared by trimming off the arms of a natural plant chromosome and adding an insertion site for large inserts (Yu et al., Proc Natl Acad Sci USA, 103:17331-6 (2006); Yu et al., Proc Natl Acad Sci USA, 104:8924-9 (2007)). The utility of engineered minichromosome platforms has been shown using Cre/lox and FRT/FLP site-specific recombination systems on a maize minichromosome where the ability to undergo recombination was demonstrated (Yu et al., Proc Natl Acad Sci USA, 103:17331-6 (2006); Yu et al., Proc Natl Acad Sci US A, 104:8924-9 (2007)). Such technologies could be applied to minichromosomes, for example, to add genes to an engineered plant. Site specific recombination systems have also been demonstrated to be valuable tools for marker gene removal (Kerbach, S. et al., Theor. Appl. Genet. 111:1608-1616 (2005)), gene targeting (Chawla, It et al., Plant Biotechnol J, 4:209-218 (2006); Choi, S. et al., Nucleic Acids Res., 28, E19 (2000); Srivastava V & Ow DW, Plant Mol. Biol. 46:561-566 (2001); Lyznik L A et al., Nucleic Acids Res., 21: 969-975 (1993)) and gene conversion (Djukanovic Vet al., Plant Biotechnol J., 4:345-357 (2006).

An alternative approach to chromosome engineering in plants involves in vivo assembly of autonomous plant minichromosomes (Carlson et al., PLoS Genet., 3:1965-74 (2007). Plant cells can be transformed with centromeric sequences and screened for plants that have assembled autonomous chromosomes de novo. Useful constructs combine a selectable marker gene with genomic DNA fragments containing centromeric satellite and retroelement sequences and/or other repeats.

Another approach useful to the described invention is Engineered Trait Loci (“ETL”) technology (U.S. Pat. No. 6,077,697; US Patent Application 2006/0143732). This system targets DNA to a heterochromatic region of plant chromosomes, such as the pericentric heterochromatin, in the short arm of acrocentric chromosomes. Targeting sequences may include ribosomal DNA (rDNA) or lambda phage DNA. The pericentric rDNA region supports stable insertion, low recombination, and high levels of gene expression. This technology is also useful for stacking of multiple traits in a plant (US Patent Application 2006/0246586).

Zinc-finger nucleases (ZFNs) are also useful for practicing the invention in that they allow double strand DNA cleavage at specific sites in plant chromosomes such that targeted gene insertion or deletion can be performed (Shukla et al., Nature, (2009); Townsend et al., Nature, (2009).

Following transformation by any one of the methods described above, the following procedures can, for example, be used to obtain a transformed plant expressing the transgenes: select the plant cells that have been transformed on a selective medium, regenerate the plant cells that have been transformed to produce differentiated plants, select transformed plants expressing the transgene producing the desired level of desired polypeptide(s) in the desired tissue and cellular location.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This is accomplished by PEG or electroporation mediated uptake, particle bombardment-mediated delivery, or microinjection. In each case the transformed cells may be regenerated to whole plants using standard techniques known in the art.

Transformation of most monocotyledon species has now become somewhat routine. Preferred techniques include direct gene transfer into protoplasts using PEG or electroporation techniques, particle bombardment into callus tissue or organized structures, as well as Agrobacterium-mediated transformation.

Plants from transformation events are grown, propagated and bred to yield progeny with the desired trait, and seeds are obtained with the desired trait, using processes well known in the art.

2. Plastid Transformation

In another embodiment the transgene is directly transformed into the plastid genome. Plastid transformation technology is extensively described in U.S. Pat. Nos. 5,451,513 to Maliga et al., 5,545,817 to McBride et al., and 5,545,818 to McBride et al., in PCT application no. WO 95/16783 to McBride et al., and in McBride et al. Proc. Natl. Acad. Sci. USA 91, 7301-7305 (1994). The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Suitable plastids that can be transfected include, but are not limited to, chloroplasts, etioplasts, chromoplasts, leucoplasts, amyloplasts, proplastids, statoliths, elaioplasts, proteinoplasts and combinations thereof.

The present invention will be further understood by reference to the following non-limiting examples.

### EXAMPLES

### Methods and Materials

The following materials and methods were used in the following examples.

### Plant Material:

Transgenic switchgrass plants, cv. Alamo, carrying the PHB pathway genes driven by the maize cab-m5 promoter (Sullivan et al., 1989; Becker et al., 1992) were used in these experiments. It has recently been shown that the control of transgene expression exerted by this promoter resulted in increased accumulation of polyhydroxybutyrate (PHB) during plant maturation (Somleva et al., 2008; US2009/0271889 A1). Primary transformants and plants propagated from them as well as their progeny producing PHB at different levels were grown under greenhouse conditions at 27° C. with a 16-hour photoperiod using supplemental lighting from Na halide lamps (200 mol/m²/s).

### Explant Source:

Tillers at the elongation stage of plant development with 2-4 nodes prior to flowering. Three types of transgenic switchgrass plants were used as donors of initial explants: i/ primary transformants obtained from transformed mature caryopsis-derived embryogenic callus cultures (Somleva et al., 2008; US2009/0271889 A1), ii/plants regenerated from immature inflorescence-derived embryogenic callus initiated from transgenic plants (this study), and iii/plants obtained from nodal segments from transgenic plants (this study).

Initiation of Tissue Cultures from Transgenic Plants:

In vitro developed panicles were obtained from the top culm node of elongating tillers from PHB producing plants following a previously published procedure for non-transformed switchgrass plants (Alexandrova et al., 1996a). For callus initiation, individual spikelets from panicles formed in tissue culture were plated on MS medium for callus initiation and growth (Denchev and Conger, 1994). Resultant embryogenic callus cultures were cultured at 28° C., in the dark and propagated by monthly transfers on to a fresh medium (Somleva, 2006).

Plants were regenerated from in vitro cultured culm nodes of elongating tillers from transgenic PHB producing plants following an established protocol for culture initiation from non-transformed switchgrass plants (Alexandrova et al., 1996b) and cultured at 28° C. with a 16 h photoperiod (cool white fluorescent bulbs, 80 μmol/m²/s).

Both types of in vitro cultures were also initiated from transgenic plants regenerated from immature inflorescence-derived callus cultures or node cultures of primary transformants.

Callus propagation and plant regeneration:

Immature inflorescence-derived callus cultures were propagated by transferring onto a fresh medium for callus growth (Denchev and Conger, 1994) every four weeks (in vitro cycles). Most of the callus cultures initiated from different donor plants were maintained for 6 months in total (six in vitro cycles).

For plant regeneration, pieces of callus were pre-weight and plated on MS medium for plant regeneration (Denchev and Conger, 1994). Cultures were incubated at 28° C. with a 16 h photoperiod (cool white fluorescent bulbs, 80 μmol/m²/s) for four weeks followed by a transfer onto a fresh regeneration medium for another four weeks. Plantlets obtained after two months were counted and their number was calculated per gram fresh weight of callus.

### PHB Analysis:

Leaf tissues (20-80 mg) from plants in tissue culture were collected, lyophilized and prepared for analysis by gas chromatography/mass spectroscopy (GC/MS) using a previously described simultaneous extraction and butanolysis procedure (Kourtz et al., 2007). For screening of PHB producing lines grown under greenhouse conditions, samples from mature leaves adjacent to the node at the base of the stem and younger still developing leaves at the top of the stem of plants grown in soil for two months were analyzed (Somleva et al., 2008). For comparative studies on the PHB spatial distribution, leaf and stem tissues from whole tillers at vegetative and reproductive developmental stages from donor and micropropagated transgenic plants were collected and analyzed by GC/MS as described previously (Somleva et al., 2008).

Re-Transformation of Immature Inflorescence-Derived Callus Cultures Initiated from Transgenic Plants:

Highly embryogenic callus cultures initiated from immature inflorescences from different PHB producing switchgrass plants were transformed with Agrobacterium tumefaciens and selected according to a previously described procedure for Agrobacterium-mediated transformation of mature caryopsis-derived callus (Somleva et al., 2002; Somleva, 2006).

### Example 1

### Transformation of Switchgrass with Multi-Gene Constructs for PHB Synthesis

Multi-Gene Constructs.

pMBXS159. This binary vector contains expression cassettes for the three gene PHB biosynthetic pathway under the control of the rice polyubiquitin 2 (rubi2) promoter (Wang et al., 2000). The PHB genes chosen for this construct include a hybrid Pseudomonas oleovorans/Zoogloea ramigera PHA synthase (Huisman et al., 2001) and the thiolase and reductase from Ralstonia eutropha (Peoples and Sinskey, 1989). Each PHA gene is fused to a plastid targeting sequence encoding the signal peptide of the small subunit of Rubisco from pea and the first 24 amino acids of the mature protein (Cashmore, 1983) as previously described (Kourtz et al., 2005). Plasmid pMBXS159 was constructed using the following multi-step procedure.

i. pMBXS124, pMBXS125, and pMBXS126. These plasmids contain rubi2 and the 3′ termination sequence of nopaline synthase (nos) and differ with respect to the restriction sites available for subsequent cloning purposes (Table 4). The rubi2 and nos fragments were isolated from template plasmids pRGL112-1 and pNEB(A) (Kourtz et al., 2007), respectively, using standard PCR techniques. The rubi2 and nos fragments were sub-cloned back into pRGL112-1 forming plasmids pMBXS124-126.

ii. pMBXS135. Plasmid pMBXS135 is a pCAMBIA3300 derived vector in which the maize hsp70 intron (Brown and Santino, 1997) has been placed between the CaMV35S promoter and bar to increase expression of the selectable marker in monocots. The maize hsp70 intron was isolated by PCR from PCR-Ready maize genomic DNA (BioChain, CA) using primers BGNP108 and BGNP109 and inserted between the CaMV35S promoter and bar gene using conventional cloning techniques.

iii. pMBXS154. Cassettes from pMBXS124, pMBXS125, and pMBXS126, containing rubi2 and nos, were sequentially transferred to pMBXS135 to form pMBXS154. Plasmid pMBXS124 was digested with Sma I and Hind III and the rubi2 nos fragment was ligated into pMBXS135 that had been previously digested with Eco RI, treated with the Klenow fragment of DNA polymerase 1, and digested with Hind III, forming intermediary plasmid pMBXS138. The rubi2 nos cassette of pMBXS125 was isolated upon digestion with Bsp EI and Hind III and the resulting fragment was inserted into the equivalent sites of pMBXS138, forming pMBXS140. The rubi2 nos cassette of pMBXS126 was isolated upon digestion with Bsr GI and Hind III and inserted into the equivalent sites of pMBXS140 forming pMBXS154.

iv. pMBXS159. The PHB biosynthetic genes, each modified with the plastid targeting signal from pea (TS), were sequentially inserted into plasmid pMBXS154 using the following multi-step procedure to form pMBXS159. A PCR fragment containing TS-phaC was isolated from pNEB(C) using conventional PCR procedures and inserted into the Avr II and Bam HI sites of pMBXS154 forming plasmid pMBXS156. A fragment containing TS-phaA-nos was isolated by PCR from plasmid pNEB(A) (Kourtz et al., 2007) and inserted into plasmid pMBXS156, which had previously been digested with Bsr GI, blunt-ended with Klenow, and digested with Bst BI, to generate pMBXS157. A fragment containing TS-phaB-nos was isolated by PCR from plasmid pNEB(B) and inserted into plasmid pMBXS157 that had been previously digested with Asc I, blunt-ended with Klenow, and digested with Hind III, to form plasmid pMBXS159.

pMBXS155. Plasmid pMBXS155 is a binary vector in which the PUB genes described above are expressed under the control of a maize chlorophyll A/B binding protein promoter (Sullivan et al., 1989). This promoter is equivalent to the cab-m5 promoter described in later work (Becker et al., 1992). In pMBXS155, the cab-m5 promoter is fused to the hsp70 intron (Brown and Santino, 1997) for enhanced expression in monocots. pMBXS155 was constructed using the following multi-step procedure.

i. pMBXS137, pMBXS143 and pMBXS144. These plasmids contain cab-m5, the hsp70 intron, and nos and differ with respect to the restriction sites available for subsequent cloning purposes. The fragment containing cab-m5 was amplified from PCR-Ready maize genomic DNA (BioChain, CA) with primers BGNP110 and BGNP111.

ii. pMBXS148. Expression cassettes from plasmids pMBXS137, pMBSX143, and pMBXS144 were sequentially inserted into plant transformation vector pMBXS135 as follows. Plasmid pMBXS137 was digested with Sma I and Hind III and the cab-m5/hsp70 nos fragment was ligated to plasmid pMBXS135 that had been previously digested with Eco RI, treated with Klenow, and digested with Hind III to create pMBXS145. This plasmid was modified by introducing an Eco RI site at the 3′ end of nos to generate pMBXS146. The cab-m5/hsp70 nos fragment of pMBXS143 was isolated with an Eco RI and Hind III digest and cloned into the Eco RI and Hind III sites of pMBXS146 to generate pMBXS147. The third cab-m5/hsp70 nos cassette was isolated from pMBXS144 by digestion with Bsr GI and Hind III was cloned into the Bsr GI and Hind III sites of pMBXS147 to create pMBXS148.

iii. pMBXS155. A fragment containing TS-phaC was isolated from pNEB(C) using conventional PCR procedures and inserted into the Avr II and Bam HI sites of pMBXS148 to form plasmid pMBXS151. Similarly, a fragment containing TS-phaA-nos was isolated by PCR from plasmid pNEB(A) (Kourtz et al., 2007) and inserted into plasmid pMBXS151, which had previously been digested with Bsr GI, blunt-ended with Klenow, and digested with Bst BI, to generate pMBXS153. Insertion of the PHB genes was completed by isolating a fragment containing TS-phaB from plasmid pNEB(B) and inserting at the Pac I and Asc I sites of pMBXS153 to create pMBXS155.

Plant Material, Transformation and Selection.

Highly embryogenic callus cultures were initiated from mature caryopses of cv. ‘Alamo’ according to established procedures (Denchev and Conger, 1994). Cultures were grown at 28° C. in the dark and maintained by monthly subcultures. After three months, callus regeneration ability was tested. Cultures capable of producing more than 350 plantlets per gram of callus were used for Agrobacterium-mediated transformation. Embryogenic cultures were infected and co-cultivated with Agrobacterium tumefaciens strain AGL1 carrying the binary vector pMBXS155 or pMBXS159 in the presence of 100 μM of acetosyringone as previously described (Somleva, 2006; Somleva et al., 2002). Cultures were selected with 10 mg/L bialaphos for 2-4 months. Resistant calluses were transferred to a regeneration medium containing 10 mg/L bialaphos (Somleva, 2006; Somleva et al., 2002). Cultures were incubated at 28° C. with a 16 h photoperiod (cool white fluorescent bulbs, 80 μmol/m²/s) for 4 to 6 weeks with biweekly subcultures. The resultant plantlets were treated with the herbicide Basta™ as described previously (Somleva, 2006; Somleva et al., 2002). Transgenic and control plants were grown in a greenhouse at 27° C. with supplemental lighting (16 hour photoperiod, Na halide lamps).

### Example 2

### Plant Regeneration from In Vitro Cultures Initiated from Transgenic Switchgrass Plants

Immature Inflorescence-Derived Embryogenic Callus Cultures.

Callus cultures were initiated from individual spikelets of in vitro developed panicles. Resultant embryogenic callus cultures were propagated by monthly transfers on to a fresh medium. At each subculture (in vitro cycle), pre-weighed callus pieces were plated on MS medium for plant regeneration.

Node Cultures.

Plants were obtained from in vitro cultured nodal segments from transgenic PHB producing switchgrass plants.

Results

The major steps of the general procedure for initiation of in vitro cultures from immature inflorescences and plant regeneration from them are shown in FIG. 1. Highly embryogenic calluses were formed from individual spikelets from panicles developed under in vitro conditions 4-6 weeks after culture initiation.

The first signs of plant regeneration were visible within 7-10 days after transfer on to a regeneration medium and green plantlets with 2-3 leaves were formed from most of the callus pieces after another 2 weeks. For further growth and development, the plantlets were incubated in large tissue culture containers with the same medium for 4 weeks followed by analysis of PHB production and transfer to soil.

The duration of the whole procedure from culture initiation to efficient regeneration of plants and their transfer to soil is about 3 months (FIG. 1). Large numbers (1,300-7,800 plants/g fresh weight of callus) of phenotypically normal plantlets were obtained from freshly initiated callus cultures from different transgenic lines.

To test the whole regeneration potential of immature inflorescence-derived cultures, some of the embryogenic callus was propagated for another 2-6 months by monthly subcultures on MS medium for callus growth followed by incubation on regeneration medium after each cycle (FIG. 1). In total, more than 42,000 plants were generated from cultures initiated from primary transformants and plants propagated from them after different in vitro cycles (Table 1). Calluses propagated for more than one month continued to produce plantlets at a high frequency within the same short period of time as freshly initiated cultures. The regenerated plants appeared normal and uniform in their growth under greenhouse conditions. A few plants with slight phenotypic changes were observed in cultures from some lines after the fifth and sixth in vitro cycles. Control cultures initiated from wild-type, non-transformed plants produced regenerants with similar phenotype after propagation for 5-6 months. No correlation between the PHB levels of the donor plants and the regeneration ability of cultures initiated from them was observed.

More than 200 plants formed directly from the axillary meristems adjacent to the culm nodes were also obtained from primary transformants and plants propagated through immature inflorescence-derived callus (Table 1). These cultures produced plants at a lower frequency compared to the highly embryogenic callus formed from in vitro cultured developing panicles. Because of their direct formation from plant meristems, these regenerants are considered true clones.

TABLE 1
Plant regeneration from in vitro cultures initiated from
immature inflorescences and nodal segments from transgenic
switchgrass plants. The number of plants analyzed for
PHB production by GC/MS is also indicated.

| | Type of | Number of | Number of | |
|---|---|---|---|---|
| Type of | in vitro | lines | regenerated plants | |
| donor plants | cultures | tested | total | analyzed |
| Primary | Inflorescence- | 14 | 30 556 | 1 203 |
| transformants | derived callus | | | |
| | Nodal | 11 | 93 | 93 |
| | segments | | | |
| Inflorescence- | Inflorescence- | 14 | 11 767 | 740 |
| derived plants | derived callus | | | |
| | Nodal | 18 | 125 | 125 |
| | segments | | | |

### Example 3

### Generation of Transgenic Switchgrass Plants with Increased PHB Levels

In vitro cultures were initiated from developing panicles or nodal segments from the following types of polymer producing plants: i/ primary transformants; ii/ plants micropropagated from them, and iii/plants obtained from immature inflorescence-derived callus or node cultures from micropropagated plants. Transgenic lines, carrying the PHB pathway genes under the control of the maize cab-m5 promoter (Somleva et al., 2008) were used as a starting material in these experiments. PHB production in most of these T₀ plants has been monitored at different stages of their growth and development under in vitro and greenhouse conditions. Plants with high, medium, and low polymer content in tissue culture or soil were included in this study.

Different approaches were used to evaluate the presence and expression of the transgenes in freshly initiated cultures and plants regenerated from them: 1/callus growth in the presence of 10 mg/L bialaphos, the concentration used for selection of transformed cultures and primary transformants (Richards et al., 2001, Somleva et al., 2002, Somleva, 2006; Somleva et al., 2008); 2/treatment of the regenerated plantlets with the herbicide Basta for functional expression of the marker gene (Somleva et al., 2002; Somleva, 2006); 3/PCR of randomly selected regenerants for confirmation of the presence of the transgenes (Somleva et al., 2008).

Results

Immature inflorescence-derived cultures from transgenic lines and plants regenerated from them were tolerant to the selecting agent bialaphos and the herbicide Basta, which also indicated sufficient levels of expression of the marker gene bar. Bialaphos at a concentration of 10 mg/L had a lethal effect on control cultures initiated from wild type, non-transformed plants. All of the analyzed plants propagated from transgenic lines had the PHB genes and the marker gene as shown by PCR.

In total, more than 1,940 transgenic plants obtained from immature inflorescence-derived callus initiated from primary transformants and plants propagated from them were analyzed for PHB production in tissue culture. Polymer content under in vitro conditions was also measured in all 218 plants regenerated directly from node cultures from the same donor lines. Plants accumulating PHB at levels up to 3-fold higher than those in the best producers among the primary transformants were identified in all populations of micropropagated transgenic plants (Table 2). The highest polymer concentrations detected were 5.25% DW and 5.66% DW PHB in plants propagated through callus and nodal segments, respectively.

Plants containing up to 6.09% DW PHB in mature leaves and 4.92% DW PHB in developing leaves were identified under greenhouse conditions (Table 2). Most of the plants propagated directly from node cultures produced polymer at levels comparable to the PHB content in the donor plants at the same developmental stage.

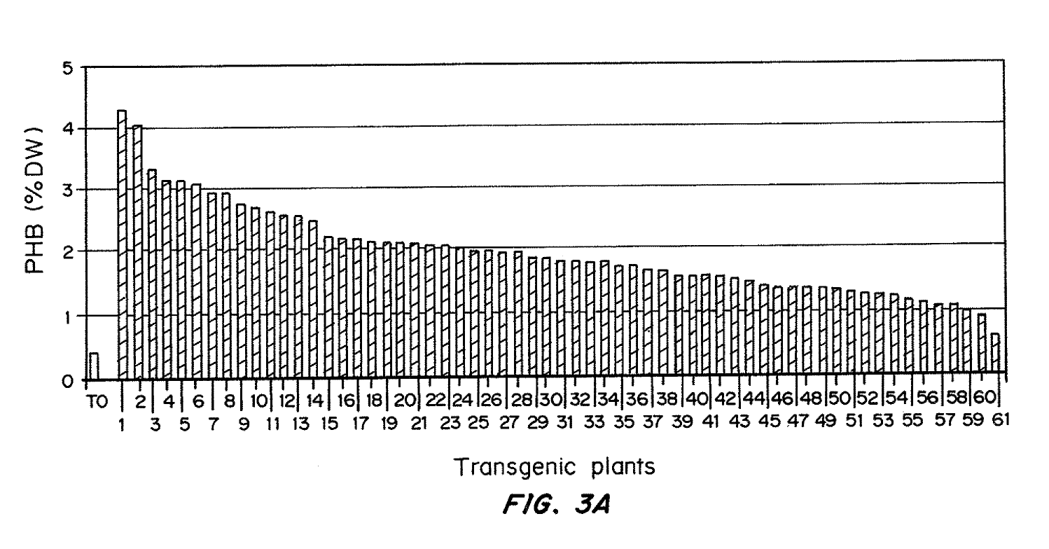

The highest polymer content was detected in plants propagated from transgenic lines with high PHB production. In total, 5,557 plants were obtained from immature inflorescence callus cultures initiated from our best characterized line 56-2a-1/3 (Somleva et al., 2008) in three independent experiments. Approximately 8% (451 plants) of them were grown in large tissue culture containers and analyzed for PHB production before transfer to soil (FIG. 2). Although the T₀ plant had only 0.45% DW PHB under in vitro conditions, plantlets accumulating more than 5.00% DW PHB in tissue culture were obtained. Some of them produced polymer at high levels in soil (see Example 4).

High producers were also identified in populations of plants propagated from cultures initiated from primary transformants with low or medium levels of polymer. For example, PHB production was monitored in 8 plants regenerated from freshly initiated cultures from a line producing 0.02% DW PHB in tissue culture and 1.09% and 0.68% DW PHB in mature and developing leaves, respectively. One of the propagated plants with 1.11% DW PHB in tissue culture accumulated 2.52% and 1.43% DW PHB in mature and developing leaves. In another transgenic line, PHB content dropped from 1.28% DW PHB in tissue culture to 0.21% and 0.05% DW PHB in mature and developing leaves, respectively, in soil. A plant propagated from inflorescence-derived cultures from this line accumulated 2.63% DW PHB in tissue culture and even more in the greenhouse—3.15% and 2.55% PHB in mature and developing leaves, respectively, from vegetative tillers.

TABLE 2
PHB production in primary transformants and in vitro propagated
plants. For GC/MS analysis in tissue culture, leaf tissues
were collected from transgenic plants regenerated from immature
inflorescence-derived callus or nodal segments at a growth
stage comparable to the stage previously used for the analysis
of the donor plants. Under greenhouse conditions, the PHB
content was measured in mature and developing leaves of vegetative
tillers from plants grown for two months.

| | | Maximum PHB content in | | |
|---|---|---|---|---|
| | | leaf tissue [% DW] | | |
| Donor | Analyzed | tissue | mature | developing |
| plants | plants | culture | leaf | leaf |
| — | Primary | 1.82 | 2.56 | 1.99 |
| | transformants | | | |
| Primary | Inflorescence | 5.25 | 6.09 | 4.92 |
| transformants | derived | | | |
| | Node culture | 4.92 | 2.45 | 1.99 |
| | derived | | | |
| Inflorescence- | Inflorescence | 5.24 | 4.90 | 3.56 |
| derived plants | derived | | | |
| | Node culture | 5.66 | 2.56 | 2.32 |
| | derived | | | |

### Example 4

### PHB Production in Plants Obtained from Immature Inflorescence-Derived Callus Cultures Initiated from a High Producer and Propagated for Six In Vitro Cycles

Highly embryogenic callus cultures were initiated from the line 56-2a-1/3 (Somleva et al., 2008) in three independent experiments. Cultures were propagated for six in vitro cycles by monthly transfers on to a fresh medium. After each in vitro cycle of four weeks, different amounts of callus (40-1,200 mg fresh weight) were used for plant regeneration. Randomly selected individual regenerants (8-40 plants per in vitro cycle) were grown in larger tissue culture containers for four weeks and sampled for GC/MS analysis.

Most of the plants accumulating more than 1.20% DW PHB were transferred to soil and grown in a greenhouse. After two months, tissues from mature and developing leaves of vegetative tillers were analyzed for PHB production (Somleva et al., 2008).

Results

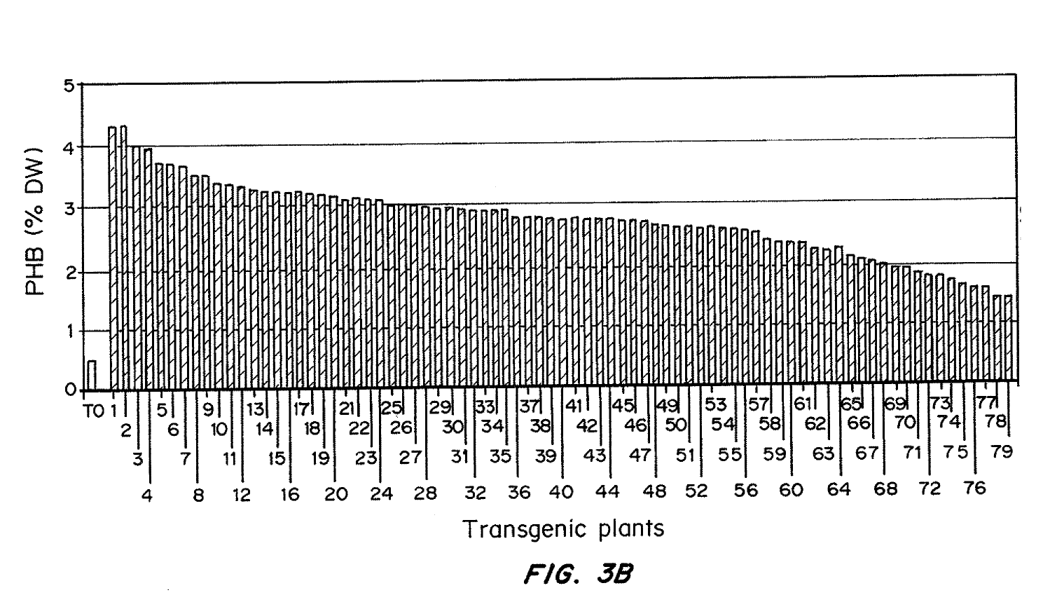

More detailed analyses were performed with plants obtained from the first and the last (sixth) in vitro cycles. PHB levels were measured in 61 plants originated from freshly initiated cultures (FIG. 3A). All of them produced polymer at higher levels (up to 9.5 times) than the primary transformant in tissue culture (0.45% DW PHB). All of the plants regenerated from callus cultures propagated for 6 months had significantly higher PHB content (1.37-4.29% DW) than the donor plant (FIG. 3B).

Under greenhouse conditions, PHB content in mature leaves of plants obtained from freshly initiated cultures was similar or higher (up to 4.29% DW) than the levels in the original line at the same developmental stage. The developing leaves contained 0.92-2.30% DW PHB. Ten plants obtained after the sixth in vitro cycle were grown in soil for 2 months for further analysis. They accumulated PHB at levels from 1.4- to 2.4-fold higher (up to 6.09% DW) than the levels in the primary transformants. Higher polymer content was also measured in the developing leaves of eight of these plants (FIG. 3C). This group of immature inflorescence-derived plants includes some of the highest PHB producers identified in this study.

### Example 5

### PHB Production in Plants Obtained from Freshly Initiated Immature Inflorescence-Derived Callus Cultures from Different Transgenic Lines

Two primary transformants with similar polymer content in soil were used for culture initiation. These plants are from the Alamo genotypes 56 and 215 previously used in transformation experiments for generation of PHB producing switchgrass lines (Somleva et al., 2008). The T₀ plants from genotype 56 described here and in the previous example are independent transformation events (represent two different transgenic lines).

Results

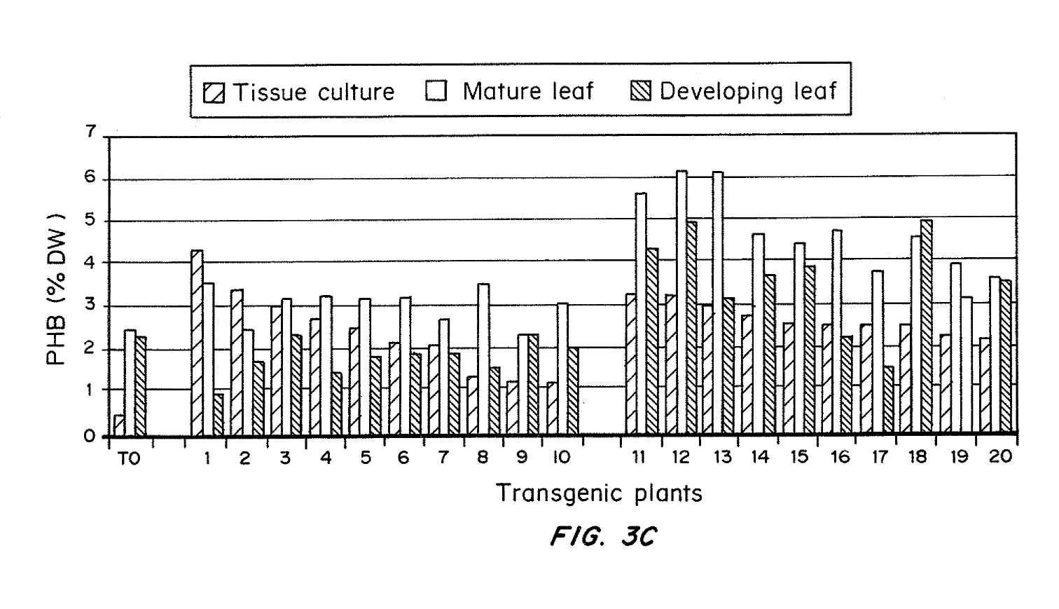

Highly embryogenic callus cultures initiated from two high producers from Alamo genotypes 56 and 215 produced 370 and 91 plants, respectively, after the first in vitro cycle. The GC/MS analysis of plants in tissue culture revealed that regenerants obtained from genotype 56 contained 0.60-2.33% DW PHB (FIG. 4A). The polymer content in all analyzed plants from genotype 215 was higher than the content in the donor T₀ plant in tissue culture (FIG. 4B).

Thirteen regenerants from Alamo genotype 56 were grown in soil for further analyses. Changes in polymer production in 12 plants had patterns similar to those observed in the primary transformant after transfer to soil (FIG. 4C). The highest PHB values were 4.55% DW and 2.75% DW in mature and developing leaves, respectively, compared to 2.29% DW and 1.94% DW in the donor plant. Only one plant showed a significant reduction in polymer production under greenhouse conditions (plant 3, FIG. 3C).

### Example 6

### PHB Production in Plants from Immature Inflorescence-Derived Callus Cultures Initiated from Transgenic Lines Propagated from Primary Transformants

In order to evaluate the effect of the origin of the donor plants on polymer production in micropropagated plants, 14 transgenic plants obtained from immature inflorescence-derived callus from primary transformants propagated for various in vitro cycles were used as sources of immature inflorescences for callus initiation and plant regeneration.

Results

More than 11,760 plants were regenerated from highly embryogenic calluses initiated from immature inflorescence-derived plants and propagated for 1-6 in vitro cycles. (The number of the cycles varied for different donor plants). All of the regenerated plants were phenotypically normal. PHB content measured in 740 plants in tissue culture varied from 0.07% DW to 5.24% DW.

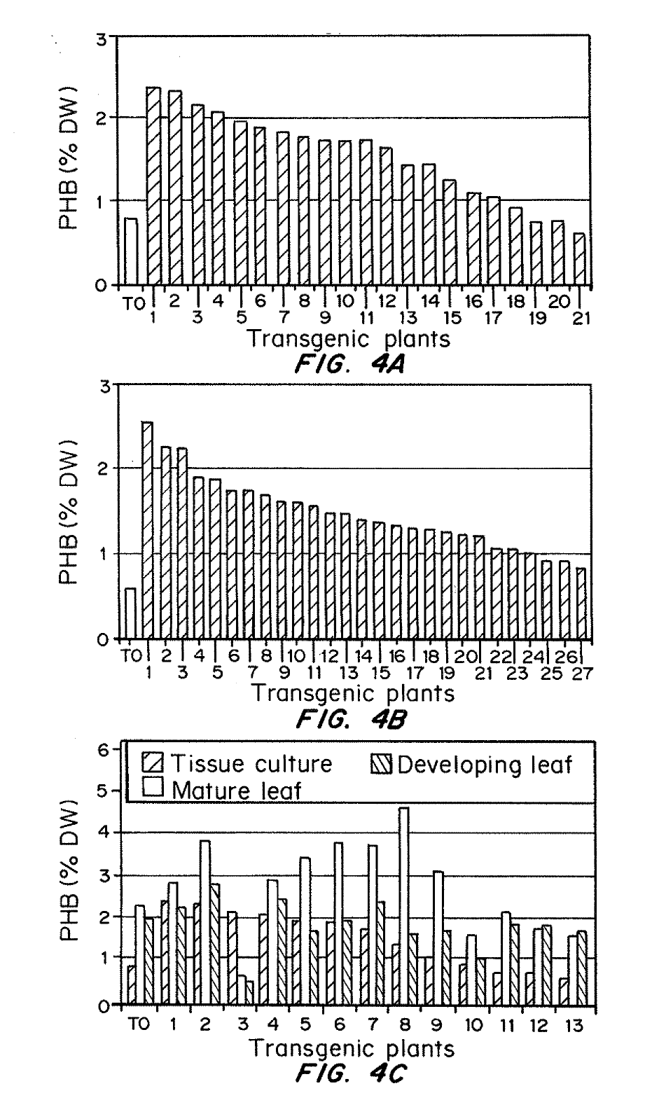

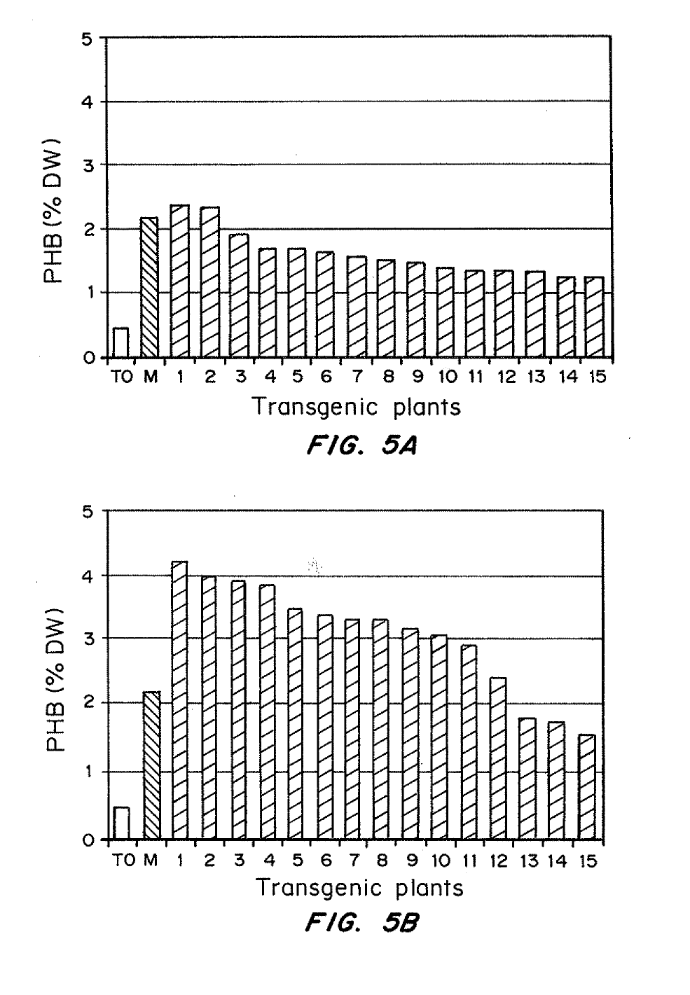

All of the regenerants obtained from freshly initiated cultures from a plant micropropagated from the high producer 56-2a-1/3 after 4 in vitro cycles had PHB content significantly higher than the content measured in the primary transformant (FIG. 5A). However, only 3 plants accumulated polymer at levels similar to the PHB content in the donor inflorescence-derived plant (M in FIG. 5). About 50% of the plants propagated from cultures maintained for 6 in vitro cycles contained significantly higher polymer levels (up to 4.20% DW) than the donor micropropagated plant (FIG. 5B).

### Example 7

### Polymer Production in Plants Regenerated from Nodal Segments from Primary Transformants and from Transgenic Plants Propagated from Them Through Immature Inflorescence-Derived Callus Cultures

Culm nodes from elongating tillers were used as an explant source for direct shoot formation from the axillary meristem. Both primary transformants and plants regenerated from immature inflorescence-derived callus were used as donor plants for culture initiation.

PHB production was analyzed in all regenerants in tissue culture. The highest producers were grown in soil for two months prior to measuring the polymer content in their mature and developing leaves.

Results

In total, 93 plants were obtained through direct regeneration from nodal segments from primary transformants. In another set of experiments, 125 plants were produced from node cultures initiated from plants originated from immature inflorescence-derived callus from primary transformants. In tissue culture, these regenerants accumulated PHB in the wide range from 0% to 5.66% DW. Under greenhouse conditions, most of them produced polymer at levels comparable to those measured in the donor plants.

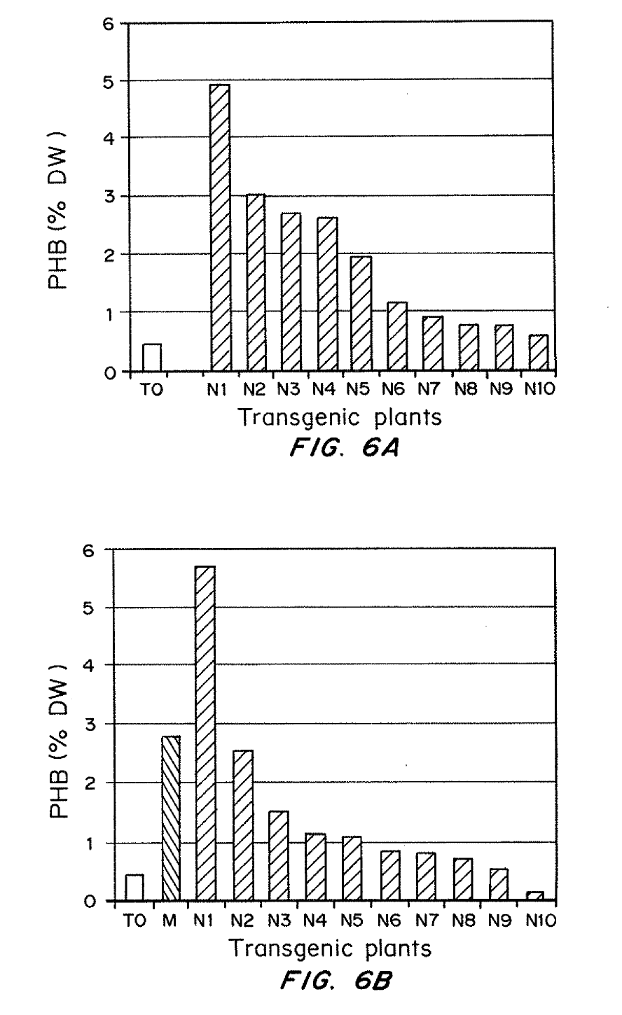

As shown in FIG. 6A, about 50% of the plants regenerated from nodal segments from the high producer 56-2a-1/3 contained significantly higher polymer content (up to 4.92% DW) than the donor plant at the same stage. Ten regenerants were obtained from nodal segments from a plant obtained from immature inflorescence cultures derived from the same primary transformant. Only one of the regenerants had a significantly higher polymer content (5.66% DW) compared to the donor plant (FIG. 6B).

These results demonstrated the possibility to obtain plants with increased PHB production through clonal micropropagation.

### Example 8

### Re-Transformation (Supertransformation) of Immature Inflorescence-Derived Callus Cultures Initiated from PHB Producing Plants

Highly embryogenic immature inflorescence-derived callus cultures initiated from two PHB producing switchgrass lines were transformed with Agrobacterium tumefaciens following previously published protocols (Somleva et al., 2002; Somleva, 2006). The transformation vector contained the marker gene hptII (conferring resistance to hygromycin) fused to the reporter gene gfp (encoding the green fluorescent protein) under the control of the constitutive promoter of the rice ubiquitin 2 gene (Wang et al., 2000). Transformed cultures and plants regenerated from them were selected with 200 mg/L hygromycin and 10 mg/L bialaphos.

The presence of the reporter gene gfp in hygromycin-resistant plantlets was confirmed by PCR using primers specific for the coding region of the gene and the amplification conditions described previously (Somleva et al., 2008). GFP fluorescence in callus cultures and plants regenerated from them was monitored microscopically at different time points during selection.

Results

As described in Example 3, cultures initiated from PHB producing switchgrass plants are resistant to bialaphos due to the expression of the marker gene bar. In order to avoid formation of any non-transformed escapes, the re-transformed cultures and regenerants from them were subjected to double selection with 200 mg/L hygromycin and 10 mg/L bialaphos.

In total, 146 pieces of immature inflorescence-derived callus were inoculated with Agrobacterium tumefaciens and selected for two months. More than 470 hygromycin-resistant plantlets were obtained from the transformed callus. All of them had the new set of transgenes (hptII and gfp) as shown by PCR.

These results demonstrate the use of cultures initiated from PHB producing switchgrass plants as target material for subsequent introduction of recombinant genes (re-transformation or supertransformation).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method for improving the characteristics of a plant the method comprising
initiating an in vitro culture from a donor plant;
regenerating a second plant from the in vitro culture, wherein the characteristics of the second plant different than the characteristics in the donor plant.

2. The method of claim 1 wherein the plant is transgenic.

3. The method of claim 1 wherein the plant is a graminaceous plant.

4. The method of claim 1 wherein the donor transgenic plant is a primary transformant.

5. The method of claim 1 wherein the donor transgenic plant was propagated from a primary transformant.

6. The method of claim 1 wherein the in vitro culture is initiated from transgenic plants regenerated from immature inflorescence-derived callus cultures or nodal segments from stably transformed plants.

7. The method of claim 1 wherein the in vitro culture is initiated from transgenic plants grown from seeds obtained from controlled crosses between transgenic plants or between transgenic and non-transgenic, wild type plants or from self-pollinated transgenic plants.

8. The method of claim 1 wherein the yield of the product from the second transgenic plant is at least two fold greater than the donor transgenic plant.

9. The method of claim 1 wherein the yield of the product from the second transgenic plant is at least three fold greater than the donor transgenic plant.

10. The method of claim 1 wherein the product comprises polyhydroxyalkanoate.

11. The method of claim 10 wherein the polyhydroxyalkanoate is polyhydroxybutyrate.

12. The method of claim 10 wherein the polyhydroxyalkanoate is a homopolymer.

13. The method of claim 10 wherein the polyhydroxyalkanoate is a copolymer.

14. The method of claim 3 wherein the plant is switchgrass, miscanthus, sugarcane, corn, arianthus, sorghum, cereals and other forage and turf grasses.

15. A method for propagating transgenic plants comprising
a) culturing immature inflorescence-derived callus cells initiated from transformed graminaceous plants;
b) dividing the callus cells into first and second portions;
c) regenerating transgenic plants from the first portion of the callus cells;
d) propagating the second portion of the callus cells to produce additional callus cells;
e) repeating steps b) and c) until a desired number of regenerated transgenic plants is achieved.

16. A method for supertransforming transgenic plants comprising
a) culturing immature inflorescence-derived callus cells initiated from transformed graminaceous plants;
b) transforming the callus cells with vector comprising a transgene different from the transgenes in the donor plant; and
e) regenerating a plant from the re-transformed callus cells.

17. The method of claim 1 wherein the donor transgenic plant is created by the method of claim 16.

18. The method of claim 17 wherein the transformed graminaceous plant is switchgrass, Miscanthus, sugarcane, corn, Arianthus, sorghum, other cereals and other forage and turf grasses.

19. The method of claim 17 wherein the transgene encodes one or more proteins involved in metabolic pathways for the synthesis of products, involved in metabolic pathways for the improvement of plant architecture and biomass yield, involved in metabolic pathways for the modification of the plant cell wall or lignin content and composition, encoding herbicide or pesticide resistance, encoding one or more hydrolytic enzyme activities, encoding proteins involved in tolerance to biotic and abiotic stress factors, involved in reduced agronomic inputs, water use efficiency or drought tollerance or in gene containment.

20. A transgenic plant produced by the method of claim 1.

21. A seed from the transgenic plant of claim 20 and a plant grown from it.

22. A biorefinery feedstock comprising the transgenic plant, plant material, or plant parts from the transgenic plant of claim 20.
